(19) United States
(12) Patent Application Publication
(10) Pub. No.: US 2026/0083784 A1
(43) Pub. Date: Mar. 26, 2026

(54) FORMULATION FOR IMPROVED BRAIN FUNCTION

(71) Applicant: Vijaykanth Tummalapally, Marietta, GA (US)

(72) Inventor: Vijaykanth Tummalapally, Marietta, GA (US)

(21) Appl. No.: 18/894,125

(22) Filed: Sep. 24, 2024

(51) Int. Cl. A61K 35/745; A61K 31/4525; A61K 35/747; A61K 36/07; A61K 36/16; A61K 36/23; A61K 36/36; A61K 36/48; A61K 36/80

(52) U.S. Cl. CPC A61K 35/745; A61K 31/4525; A61K 35/747; A61K 36/07; A61K 36/16; A61K 36/23; A61K 36/36; A61K 36/48; A61K 36/80

(57) ABSTRACT

The invention is a formulation of probiotics and herbal supplements that preserve or improve brain function by preserving or enhancing nerve and brain health and operation.

### TECHNICAL FIELD

The invention is a formulation for improving brain function.

### BACKGROUND OF INVENTION

Maintaining optimal brain function involves addressing multiple factors, including cardiovascular health, nutrition, sleep, mental health, stress management, social engagement, and lifestyle choices. By focusing on these modifiable factors, individuals can take proactive steps to support their brain health and cognitive abilities throughout life.

Medical research has found that ingesting probiotics and other nutritional supplements can positively affect brain function throughout one's life.

### BRIEF DESCRIPTION OF INVENTION

The invention disclosed is formulation of probiotics and other supplements that are safely digested and are known to positively affect brain function.

The combination of specific probiotics by quantity, and other supplements by weight, is believed to be a novel combination.

Combined in a single, dissolvable capsule, and in safe dosage levels, this formulation is meant to be a daily, dietary, supplement aimed at preserving or improving brain and mental function.

### BRIEF DESCRIPTION OF DRAWINGS

FIG. 1 shows a table of probiotic formulation constituents by quantity of each.

FIG. 2 shows a table of non-probiotic constituents by weight.

### DETAILED DESCRIPTION OF INVENTION

The human nervous system is a complex system comprising brain, sensors, and nerves. Sensory input, such as that produced by light on the human retina, is conveyed via optic nerves to the portion of the brain devoted to receiving and reacting to such input by creating mental images. Excess incoming luminosity may, in turn, cause the brain to issue signals back to the eye to cause constriction of the pupil to reduce the amount of photons entering the eye and impinging on the retina.

Many of the things we associate with nervous-system function depend upon this bidirectional process of sensory inputs, brain interpretation, and nerves conveying brain signals to affected body parts. For optimal function, the sensors, nerves and brain must be in optimal physical and operational condition.

As in all other bodily cells, sensor, brain and nerve cells must receive adequate oxygen and nutrients to remain viable and productive. Where dietary intake lacks certain nutrients, the health of sensors, brain and nerves may be affected.

Based on research probing the effects of specific probiotics and other herbal supplements on the health of the nervous system and that of brain function, the following combination of these probiotics and other herbal supplements has been combined to create a formulation that combines the individual benefits of each constituent into a daily supplement that is safe to digest and shown to positively affect brain function.

Regarding the probiotics, the combination comprises two categories of probiotics: bifidobacteria and lactobacilli. This blend of probiotics, as shown by research evidence, affects brain function and memory by modulating the gut-brain axis, a bidirectional communication system between the gut microbiota and the central nervous system. These probiotics balance gut microbiota, reduce inflammation and increase production of short-chain fatty acids, such as butyrate. They also influence production of neurotransmitter, such as serotonin and gamma-aminobutyric acid which affect mood and cognition.

Regarding the other herbal supplements that comprise this formulation, hericium erinaceous, also known as lion's mane, stimulates the production of nerve growth factor, a protein associated with the growth, maintenance, and survival of neurons. These benefits are attributed to the two principal constituents: hericenones and erinacines. By promoting neurogenesis and myelination, hericium erinaceous helps support brain plasticity, believed essential for learning, memory, and overall cognitive function. Additionally, hericium erinaceous' antioxidant properties protect neurons from oxidative stress, which can contribute to cognitive decline. Clinical studies suggest that hericium erinaceous can improve memory and cognitive function, particularly in individuals experiencing mild cognitive impairment.

Ginkgo biloba extract, standardized to 24% flavonol glycosides and 6% terpene lactones, enhances brain function and memory through multiple mechanisms. The flavonol glycosides act as antioxidants, protecting brain cells from oxidative damage, while the terpene lactones improve blood flow to the brain by dilating blood vessels and reducing platelet aggregation. This increased cerebral circulation ensures better oxygen and nutrient delivery to neurons, supporting cognitive processes. Ginkgo biloba also modulates neurotransmitter activity, particularly acetylcholine, which is associated with memory and learning. Clinical studies have shown that ginkgo biloba can improve memory, attention, and cognitive function, especially in individuals with age-related cognitive decline or conditions like Alzheimer's disease.

Mucuna pruriens extract, standardized to 20% L-DOPA, enhances brain function, memory, and motivation by directly supplying L-DOPA, a precursor to the neurotransmitter dopamine. Dopamine plays a key role in regulating mood, motivation, and cognitive functions such as attention and learning. By increasing dopamine levels in the brain, mucuna pruriens improves mental clarity, focus, and memory retention. Additionally, higher dopamine levels boost motivation and drive, making it easier to stay engaged and productive.

Bacopa monnieri also known as Brahmi, standardized to 45% bacosides, enhances brain function and memory by promoting synaptic communication and supporting neuroplasticity. Bacosides, the active compounds, boost the production and repair of neurons and synapses, which are critical for learning and memory retention. Additionally, Bacopa has antioxidant properties that protect brain cells from oxidative stress and may reduce inflammation, which can contribute to cognitive decline. It also modulates neurotransmitter levels, particularly acetylcholine and serotonin, improving memory recall and reducing anxiety, which can further enhance cognitive performance. Clinical studies have shown that bacopa monnieri improves cognitive functions like memory, attention, and information processing.

Beta vulgaris, also known as beet root, enhances brain function and memory primarily through its high nitrate content. Once ingested, these nitrates are converted into nitric oxide (NO) in the body, a molecule that promotes vasodilation, increasing blood flow and oxygen delivery to the brain. Improved cerebral blood flow enhances cognitive functions, including memory, focus, and mental clarity. Additionally, the increased nitric oxide levels support neuroplasticity, the brain's ability to form new neural connections, which is vital for learning and memory retention. Beet root extract also possesses antioxidant properties, protecting brain cells from oxidative stress and inflammation, further supporting cognitive health.

Centalla asiatica, also known as gotu kola enhances brain function and memory through several mechanisms. Asiaticosides, the active compounds, promote neurogenesis by stimulating the growth of new neurons and enhancing synaptic plasticity, which are critical for learning and memory retention. Additionally, centalla asiatica improves cerebral circulation, allowing better oxygen and nutrient delivery to brain cells, which supports cognitive function. Its antioxidant properties protect neurons from oxidative stress and inflammation, further preserving cognitive abilities. Centella asiatica also modulates the release of neurotransmitters such as acetylcholine, which supports memory and learning processes.

Piperine, an active compound in black pepper, enhances the bioavailability of various herbs by inhibiting enzymes that metabolize them, such as CYP3A4 and P-glycoprotein. This inhibition allows higher concentrations of the active compounds from these herbs, like bacosides in bacopa, L-DOPA in mucuna pruriens, terpene lactones from ginkgo, and tannins in triphala, to remain in the bloodstream for longer periods. Additionally, piperine increases the permeability of the intestinal lining, facilitating better absorption of these compounds into the bloodstream. By slowing down their breakdown and enhancing their absorption, piperine helps the body utilize the active ingredients of these herbs more effectively, leading to improved therapeutic effects, including enhanced cognitive function and memory.

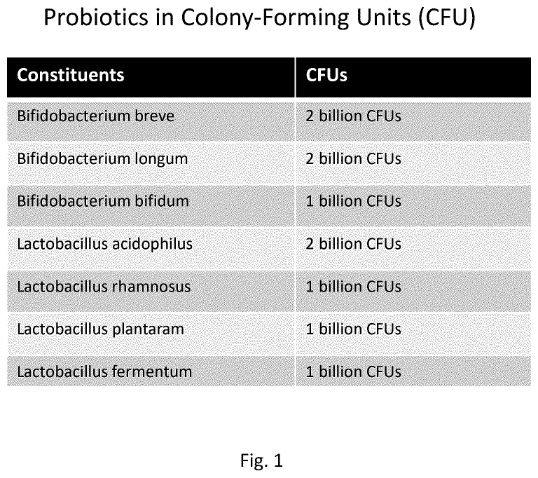

The preferred embodiment of the invention formulation is shown, in FIG. 1, for the probiotics. The amounts shown are based on colony-forming units (CFUs) which by definition estimate the number of viable cells in a sample. In this instance, the formulation quantities indicate the number of live bacteria per dose.

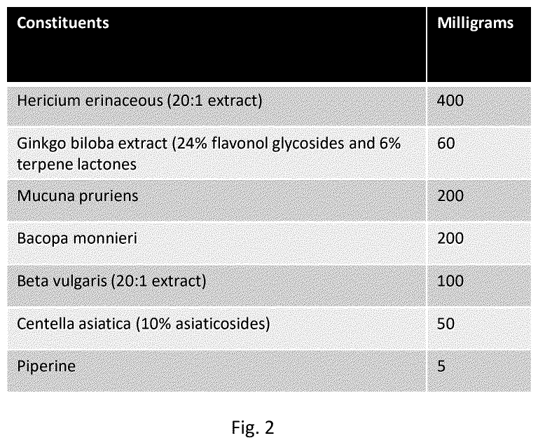

The remaining supplements in the formulation are shown in FIG. 2, and are indicated by weight in milligrams.

This is the preferred embodiment. Other embodiments may alter the CFUs and weights of these supplements. The novelty relates to the specific probiotics and accompanying supplements comprising the formulation and is not dependent upon specific quantities and weights. The quantities and weights may vary through a range of values and the scope of claims should not be limited to the preferred embodiment values.

What is claimed is a formulation comprising:

1. A combination of probiotics comprising:
bifidobacterium breve;
bifidobacterium longum;
bifidobacterium bifidum;
lactobacillus acidophilus;
lactobacillus rhamnosus;
lactobacillus plantaram; and
lactobacillus fermentum.

2. A claim as in claim 1 further comprising:
hericium erinaceous;
ginkgo biloba extract;
mucuna pruriens;
bacopa monnieri;
beta vulgaris;
centella asiatica; and
piperine.

3. A claim as in claim 1 wherein:
the quantity of the bifidobacterium breve is essentially two billion CFUs;
the quantity of the bifidobacterium longum is essentially two billion CFUs;
the quantity of the bifidobacterium bifidum is essentially one billion CFUs;
the quantity of the lactobacillus acidophilus is essentially two billion CFUs;
the quantity of the lactobacillus rhamnosus is essentially one billion CFUs;
the quantity of the lactobacillus plantaram is essentially one billion CFUs; and
the quantity of the lactobacillus fermentum is essentially one billion CFUs.

4. A claim as in claim 2 wherein:
the weight of the hericium erinaceous is essentially 400 milligrams;
the weight of the ginkgo biloba extract is essentially 60 milligrams;
the weight of the mucuna pruriens is essentially 200 milligrams;
the weight of the bacopa monnieri is essentially 200 milligrams;
the weight of the beta vulgaris is essentially 100 milligrams;
the weight of the centella asiatica is essentially 50 milligrams; and
the weight of the piperine is essentially 5 milligrams.
